(19) United States
(12) Patent Application Publication
(10) Pub. No.: US 2024/0180584 A1
(43) Pub. Date: Jun. 6, 2024

(54) HIGH SPEED CHRONIC TOTAL OCCLUSION CROSSING DEVICES

(71) Applicant: Avinger, Inc., Redwood City, CA (US)

(72) Inventors: Himanshu N. PATEL, San Jose, CA (US); John B. SIMPSON, Woodside, CA (US); Ryan RADJABI, Campbell, CA (US)

(21) Appl. No.: 18/439,575

(22) Filed: Feb. 12, 2024

Related U.S. Application Data
(63) Continuation of application No. 17/450,658, filed on Oct. 12, 2021, now Pat. No. 11,931,061.
Application No. 17/450,658 is a continuation of application No. 16/516,093, filed on Jul. 18, 2019, now Pat. No. 11,147,583.
Application No. 16/516,093 is a continuation of application No. 15/324,325, filed on Jan. 6, 2017, now Pat. No. 10,357,277.
(60) Provisional application No. 62/073,850, filed on Oct. 31, 2014.
(60) Provisional application No. 62/022,101, filed on Jul. 8, 2014.

(51) Int. Cl. A61B 17/3207; A61B 1/00; A61B 1/005; A61B 1/313; A61B 17/00; A61B 17/22; A61B 90/00

(52) U.S. Cl. CPC A61B 17/320758; A61B 1/00066; A61B 1/00073; A61B 1/00165; A61B 1/005; A61B 1/3137; A61B 17/00234; A61B 2017/00309; A61B 2017/00455; A61B 2017/00907; A61B 2017/22094; A61B 2017/320775; A61B 2090/3614; A61B 2090/3735; A61B 2090/3983

(57) ABSTRACT

An occlusion crossing device includes an outer shaft, an inner shaft, an optical fiber, and a handle attached to the inner shaft and the outer shaft. The inner shaft extends within the outer shaft. The inner shaft includes a drill tip at a distal end thereof. The optical fiber extends within the inner shaft substantially along a central axis of the inner shaft. The distal tip of the optical fiber is attached to the drill tip. The handle is configured to rotate the inner shaft and drill tip at speeds of greater than 500 rpm.

### CROSS REFERENCE TO RELATED APPLICATIONS

This application is a continuation of U.S. patent application Ser. No. 17/450,658, filed Oct. 12, 2021, titled “HIGH SPEED CHRONIC TOTAL OCCLUSION CROSSING DEVICES,” now U.S. Patent Application Publication No. 2022/0039828, which is a continuation of U.S. patent application Ser. No. 16/516,093, filed on Jul. 18, 2019, titled “HIGH SPEED CHRONIC TOTAL OCCLUSION CROSSING DEVICES,” now U.S. Pat. No. 11,147,583, which is a continuation of U.S. patent application Ser. No. 15/324,325, filed on Jan. 6, 2017, titled “HIGH SPEED CHRONIC TOTAL OCCLUSION CROSSING DEVICES,” now U.S. Pat. No. 10,357,277, which is a U.S. National Phase Application Under 35 U.S.C. § 371 of International Application No. PCT/US2015/039585, filed on Jul. 8, 2015, titled “HIGH SPEED CHRONIC TOTAL OCCLUSION CROSSING DEVICES,” now International Publication No. WO 2016/007652, which claims priority to U.S. Provisional Patent Application No. 62/073,850, titled “HIGH SPEED CHRONIC TOTAL OCCLUSION CROSSING DEVICES,” filed on Oct. 31, 2014 and U.S. Provisional Patent Application No. 62/022,101, titled “HIGH SPEED CHRONIC TOTAL OCCLUSION CROSSING DEVICES,” filed on Jul. 8, 2014, the entire contents of each are incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Peripheral artery disease (PAD) and coronary artery disease (CAD) affect millions of people in the United States alone. PAD and CAD are silent, dangerous diseases that can have catastrophic consequences when left untreated. CAD is the leading cause of death for in the United States while PAD is the leading cause of amputation in patients over 50 and is responsible for approximately 160,000 amputations in the United States each year.

Coronary artery disease (CAD) and Peripheral artery disease (PAD) are both caused by the progressive narrowing of the blood vessels most often caused by atherosclerosis, the collection of plaque or a fatty substance along the inner lining of the artery wall. Over time, this substance hardens and thickens, which may interfere with blood circulation to the arms, legs, stomach and kidneys. This narrowing forms an occlusion, completely or partially restricting flow through the artery. Blood circulation to the brain and heart may be reduced, increasing the risk for stroke and heart disease.

Interventional treatments for CAD and PAD may include endarterectomy and/or atherectomy. Endarterectomy is surgical removal of plaque from the blocked artery to restore or improve blood flow. Endovascular therapies such as atherectomy are typically minimally invasive techniques that open or widen arteries that have become narrowed or blocked. Other treatments may include angioplasty to open the artery. For example, a balloon angioplasty typically involves insertion of a catheter into a leg or arm artery and positioning the catheter such that the balloon resides within the blockage. The balloon, connected to the catheter, is expanded to open the artery. Surgeons may then place a wire mesh tube, called a stent, at the area of blockage to keep the artery open.

Such minimally invasive techniques (e.g., atherectomy, angioplasty, etc.) typically involve the placement of a guidewire through the occlusion. Using the guidewire, one or more interventional devices may be positioned to remove or displace the occlusion. Unfortunately, placement of the guidewire, while critical for effective treatment, may be difficult. In particular, when placing a guidewire across an occlusion, it may be difficult to pass the guidewire through the occlusion while avoiding damage to the artery. For example, it is often difficult to prevent the guidewire from directing out of the lumen into the adventitia and surrounding tissues, potentially damaging the vessel and preventing effective treatment of the occlusion.

As a result, occlusion-crossing devices, intended to assist in the passing of the guidewire through the occlusion, have been developed. Many of the devices, however, are ill equipped to be used with imaging, thereby making placement of the guidewire cumbersome and difficult. Moreover, many of the occlusion-crossing devices are too large to be used in small-diameter peripheral arteries or in coronary arteries.

Accordingly, occlusion crossing catheter devices designed to address some of these concerns are described herein.

### SUMMARY OF THE DISCLOSURE

Described herein are occlusion-crossing devices having a low profile and a distal drill tip. In some embodiments, an articulating feature can provide for steering or directionality of the device. In some embodiments, an inner shaft can be removable from an outer shaft.

In general, in one embodiment, an occlusion crossing device includes an outer shaft, an inner shaft, an optical fiber, and a handle attached to the inner shaft and the outer shaft. The inner shaft extends within the outer shaft. The inner shaft includes a drill tip at a distal end thereof. The optical fiber extends within the inner shaft substantially along a central axis of the inner shaft. The distal tip of the optical fiber is attached to the drill tip. The handle is configured to rotate the inner shaft and drill tip at speeds of greater than 500 rpm.

This and other embodiments can include one or more of the following features. The inner shaft and optical fiber can be removable from the outer shaft. The handle can include a luer lock configured to lock and unlock the inner shaft relative to the outer shaft. The outer shaft can include an articulating feature configured to allow the outer shaft to bend. The articulating feature can be activated by moving the inner shaft along the central axis relative to the outer shaft. The articulating feature can include a backbone and a plurality of circumferential cuts. The inner shaft can include an annular member configured to engage with an inner lip of the outer shaft to bend the outer shaft when the inner shaft is pushed distally. The inner shaft can include an annular member configured to engage with an inner lip of the outer shaft to bend the outer shaft when the inner shaft is pulled proximally. The outer shaft can include a preformed bend therein. The outer shaft can further include a marker positioned with respect to the preformed bend such that an orientation of the outer shaft can be determined during imaging. The outer shaft can include a transparent distal portion configured to allow imaging with the optical fiber therethrough. The handle can be configured to rotate the inner shaft and drill tip at speeds of greater than 1,000 rpm. The handle can be configured to rate the inner shaft and drill tip at speeds of greater than 500 rpm such that images can be generated from the optical fiber at a rate of greater than or equal to 8 frames per second. The optical fiber can be a common path optical coherence tomography fiber. The drill tip can include a plurality of spiral cutting edges. The drill tip can be a substantially smooth frusto-conical tip. The imaging device can further include a monorail guidewire lumen extending along the outer shaft. An outer diameter of the outer shaft can be less than 0.08 inches.

In general, in one embodiment, a method of crossing an occlusion includes: (1) inserting a device into a vessel having an occlusion therein; (2) rotating an inner shaft of the device relative to an outer shaft of the device such that a drill tip on the inner shaft drills through the occlusion; and (3) generating images with an optical fiber extending through the inner shaft at a rate of greater than or equal to 8 frames per second while rotating the inner shaft.

This and other embodiments can include one or more of the following features. The method can further include removing the inner shaft from the outer shaft, and inserting a guidewire through the outer shaft. The method can further include bending a distal end of the device in order to steer the device through the vessel. Bending the distal end can comprise pushing or pulling on the inner shaft. The method can further include orienting a bend in the outer shaft in a desired direction. The method can further include using a marker on the device to orient the bend. Rotating the inner shaft can comprise rotating at more than 500 rpm.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-3B show an occlusion crossing device having an articulating feature.

FIGS. 4A-5C show an occlusion crossing device having separable inner and outer shafts.

FIGS. 6A and 6B are exemplary block diagrams of drive systems for the catheters described herein.

FIGS. 7A-7B show an exemplary method for detecting the position of the driveshaft of a catheter.

FIGS. 8A-8B show articulation of an occlusion crossing device having an articulating feature and separable inner and outer shafts.

FIGS. 9A and 9B show the inner shaft of the occlusion crossing device of FIGS. 8A-8B positioned inside of the outer shaft for cutting and imaging.

FIG. 10 shows the outer shaft of the occlusion crossing device of FIGS. 8A-8B with the inner shaft removed.

FIGS. 11A and 11B show an exemplary handle for use with the occlusion crossing device of FIGS. 8A-8B.

FIGS. 12 and 13 show another exemplary handle for use with the occlusion crossing device of FIGS. 8A-8B.

FIGS. 14A-14C show the distal portion of another embodiment of an occlusion crossing device.

FIG. 15 shows the occlusion crossing device FIGS. 1A-3B with a monorail guidewire lumen.

### DETAILED DESCRIPTION

Described herein are occlusion-crossing devices having a low profile so as to be usable in small-diameter arteries and coronary arteries, e.g., through a 5 French catheter or smaller. In general, the devices described herein can have on-board imaging, such as optical coherence tomography (OCT) imaging. The optical fiber for OCT imaging can extend substantially along the central axis of the device, thereby decreasing the profile of the device and allowing for rotation at high speeds. The devices can also include a rotatable pointed tip, allowing for forward drilling. In some embodiments, the device can include an articulating distal end to enable steering of the device.

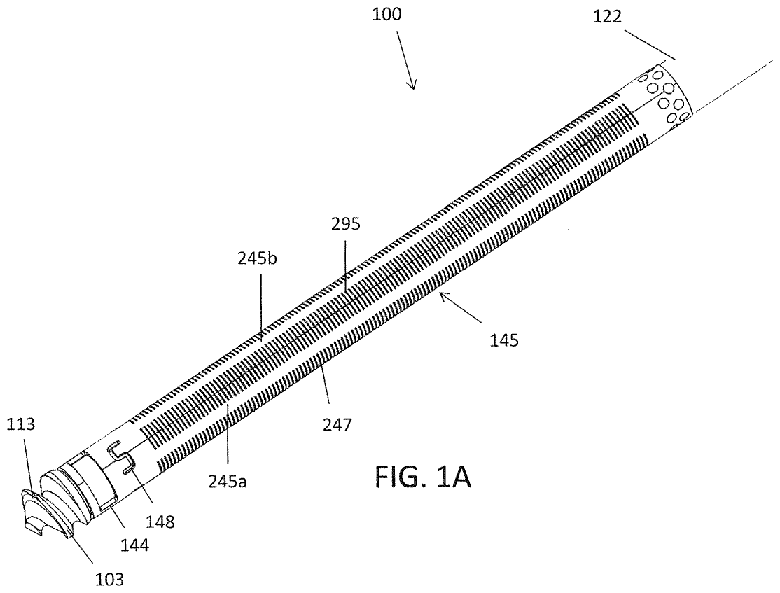

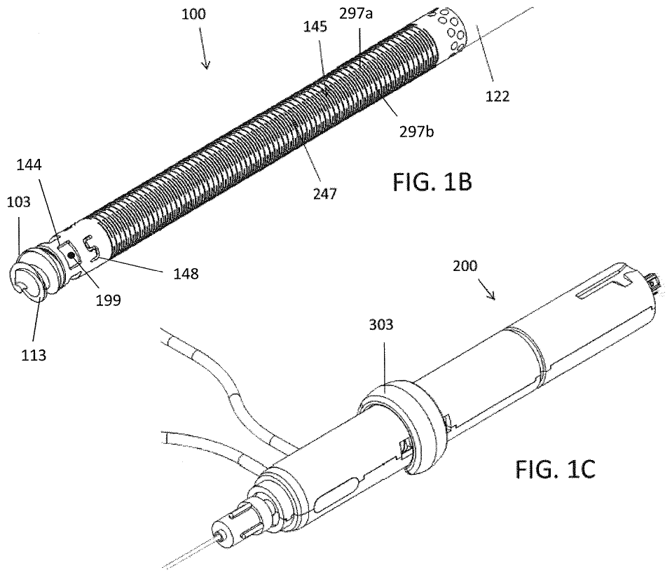

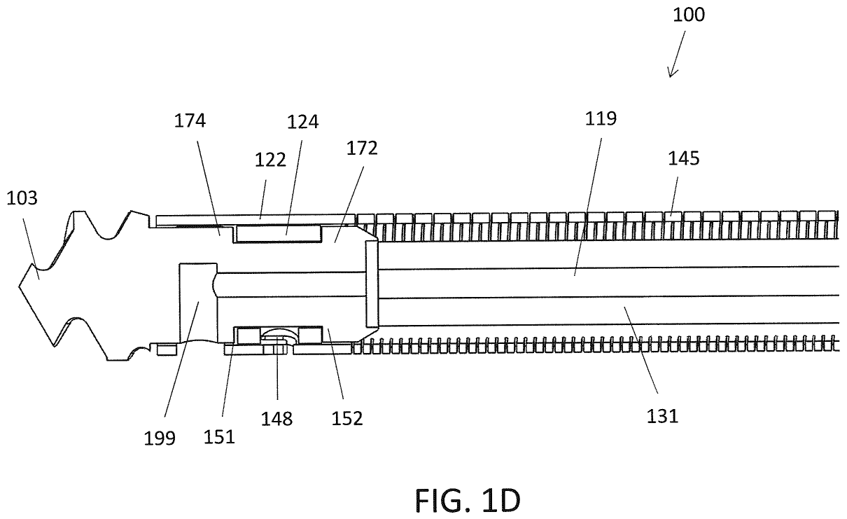

Referring to FIGS. 1A-3B, in one embodiment, an exemplary catheter 100 includes an outer shaft 122 and an inner driveshaft 131 connected to a distal tip 103. The elongate outer shaft 122 can be hollow and can have an inner diameter of approximately 1 mm and an outer diameter of approximately 1.5 mm. In some embodiments, the outer shaft 122 can have a coiled construction, whereby the coils are wound by laying one coil over another. For example, the shaft 122 can include at least two coil layers. Further, the coil layers can be counter-wound, such that one coil layer, such as the inner coil layer, has a left hand lay and another layer, such as the outer coil layer, has a right hand lay. The coil can provide torque in the direction that tightens the outer layer, cinching down on the inner layer. A third counter wound coil can be added to generate torque in both directions. In another embodiment, the shaft 122 is made of a braided wire reinforced polymeric shaft. In yet another embodiment, the shaft 122 can be a laser-cut tube. The outer shaft 122 can further include one or more imaging windows 144 at a distal end thereof.

A bushing 124 (see FIG. 1D) can be attached to the shaft 122, such as through a tab and slot mechanism 148. The bushing 124 can act as a bearing surface relative to the inner shaft or tip 103. Further, the bushing 124 can include edges or lips 151, 152 on either side configured to interact with the inner driveshaft 131 or the tip 103, as discussed further below.

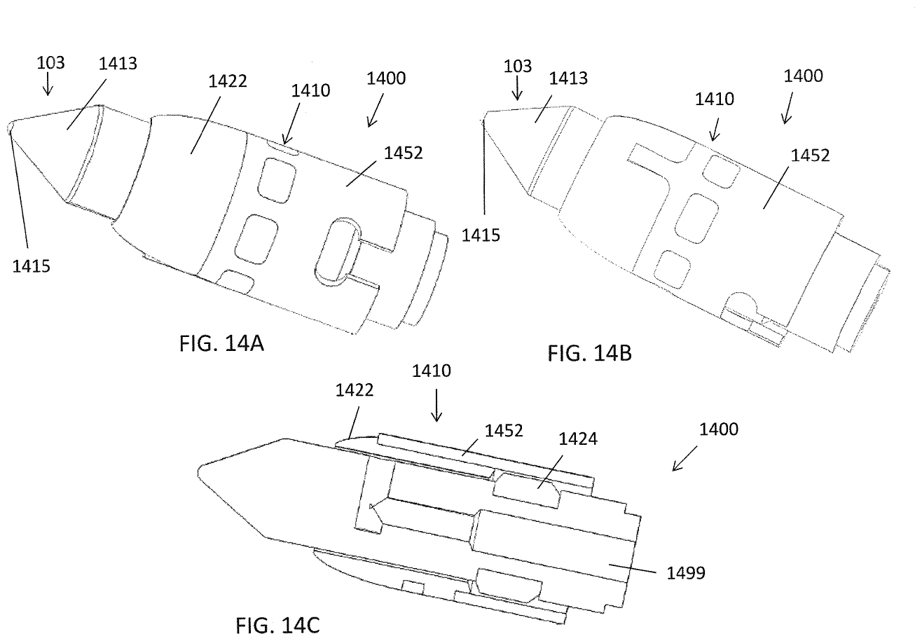

The tip 103 can be configured, for example, to separate, dissect, or shred tissue. In some embodiments, the tip 103 can include sharp spiraling flutes 113 that come to a point in the center of the device. Further, the flutes 113 can be angled such that they have sharper edges when rotated in one direction than in another direction. As a result, the tip 103 with flutes 113 can have an active and passive modes depending upon the direction of rotation of the tip 103. In passive mode, the tip 103 with flutes 113 can be less aggressive, providing blunt dissection of tissue. In active mode, the tip 103 with flutes 113 can be more aggressive, providing cutting and auguring to make its way through harder material. In some embodiments, as described further below with respect to FIGS. 14A and 14B, the distal tip 103 can have a smooth angled surface that is non-fluted.

The inner driveshaft 131 (see FIG. 1D) can be connected to the distal tip 103 and can extend down the center of the outer shaft 122. The inner driveshaft 131 can be configured to rotate in either a single direction or in both the clockwise and counterclockwise directions so as to rotate the tip 103 relative to the shaft 122 (about the bushing 124) in either a single direction or in the clockwise or counterclockwise direction. Annular rings 174, 172 can be positioned around a distal portion of the inner driveshaft 131 and/or the tip 103. The rings 174, 172 can be positioned against the edges 151, 152 of the bushing 124. The annular bushing 124 can allow relative rotation of the inner driveshaft 131 relative to the bushing 124 while preventing axial movement (and allowing for articulation in some embodiments, as described further below).

In some embodiments, a distal portion of the outer shaft 122 can include an articulating feature 145. As shown in FIGS. 1A and 1B, the articulating feature 145 can include one or more backbones 245a,b and a series of circumferential cuts 247 and 295. The one or more backbones can be positioned on only one side of the catheter (e.g., span less than 180 degrees, less than 150 degrees, or less than 90 degrees). In some embodiments, and as shown in FIG. 1A, a series of small circumferential cuts 295 can extend between the two backbones 245a,b in order to provide added flexibility during bending. The circumferential cuts 247, 295 can be configured as incomplete rings or spirals about the outer shaft 122. Referring to FIG. 1B, in some embodiments, the circumferential cuts 247 can include one or more breaks 297a,b therein designed to provide additional tensile strength and compression resistance for the articulating feature 145.

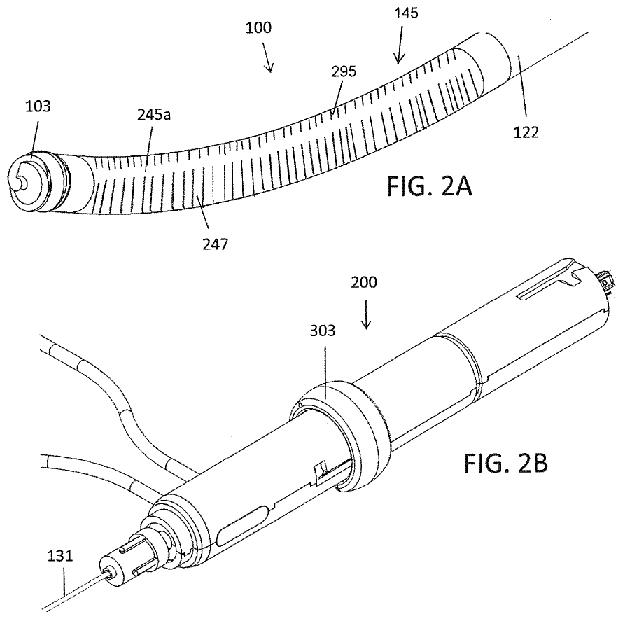

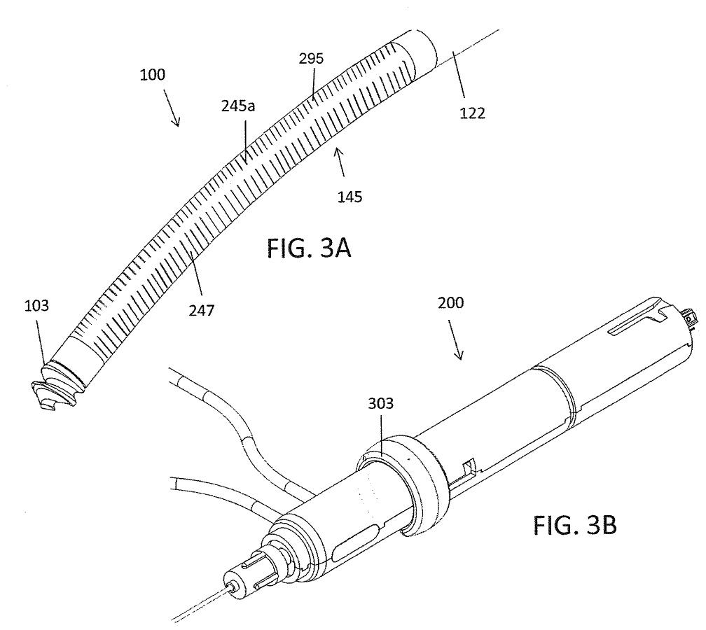

The articulating feature 145 can be attached to the inner driveshaft 131 such that movement of the driveshaft 131 can activate the articulating feature. Further, in some embodiments, a handle 200 (see FIGS. 2B and 3B) can be used to activate movement of the driveshaft 131.

Referring to FIGS. 2A-2B, as the driveshaft 131 is pushed distally, the annular ring 172 can push distally on the proximal lip 152 of the bushing 124 (see FIG. 1D), causing the circumferential cuts 247 to spread apart or open while the backbones 245a,b maintain their length (and the circumferential cuts 295 move closer together). As a result, the articulating feature 145 can bend towards the backbones 245a,b. As shown in FIG. 2B, this bending mechanism can be activated on the handle 200, such as by moving a ring 303 distally and/or pushing or moving a button or lever.

Likewise, referring to FIGS. 3A-3B, as the driveshaft 131 is pulled proximally, the annular ring 174 can hit the distal lip 151 of the bushing 124. As further distal force is applied by the driveshaft 131, the circumferential cuts 247 can begin to move closer together and/or the material between the cuts 247 can overlap while the backbones 245a,b maintain their length (and the cuts 295 move further apart). As a result, the articulating feature 145 can bend towards the circumferential cuts 247 and away from the backbones 245a,b. As shown in FIG. 3B, this bending mechanism can be activated on the handle 200, such as by moving the ring 303 proximally and/or pushing or moving a button or lever.

The bending movement of the articulating feature 145 can advantageously allow the device 100 to be steered when used in the vessel, such as for re-entry if the tip extends out of the occlusion or lumen. In some embodiments, the catheter 100 can be configured to bend in only one direction by either pushing or pulling on the driveshaft 131 and return to the straight configuration shown in FIG. 1A by movement of the driveshaft 131 in the opposite direction.

The catheter 100 can further include an imaging element 199 attached to the driveshaft 131 and configured to rotate therewith. The imaging element 199 can be the distal end of an OCT fiber 119 extending down the center of the driveshaft 131. The imaging element 199 can provide imaging (through windows 144) as the catheter 100 is used in the vessel, thereby assisting in occlusion crossing.

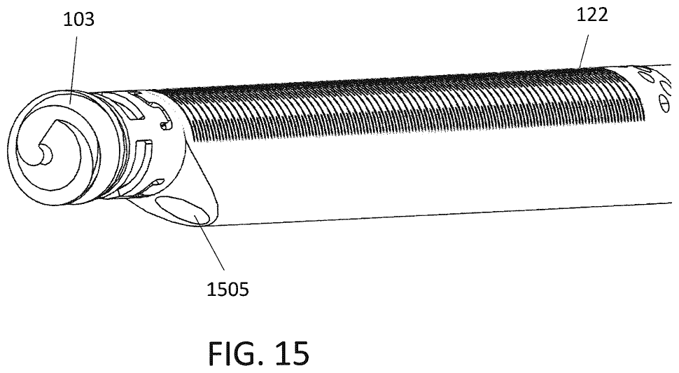

Referring to FIG. 15, in some embodiments, a monorail guidewire lumen 1505 can extend along the outer shaft 122. The guidewire lumen 1505 can run, for example, between the two backbones 245a,b so as to not add additional stiffness to the flexible area with the circumferential cuts 247.

In some embodiments, the catheter 100 can be used with a sheath. The sheath can be hollow and include a hemostasis valve attached at the proximal end with a flush port on the side to facilitate flushing through the sheath. The sheath can also facilitate guidewire placement to the target site, particularly for embodiments of the catheter 100 that do not include a monorail guidewire lumen. That is, the catheter 100 can be used to cross the occlusion, the sheath can be placed thereover, the device removed, and then the guidewire can be introduced.

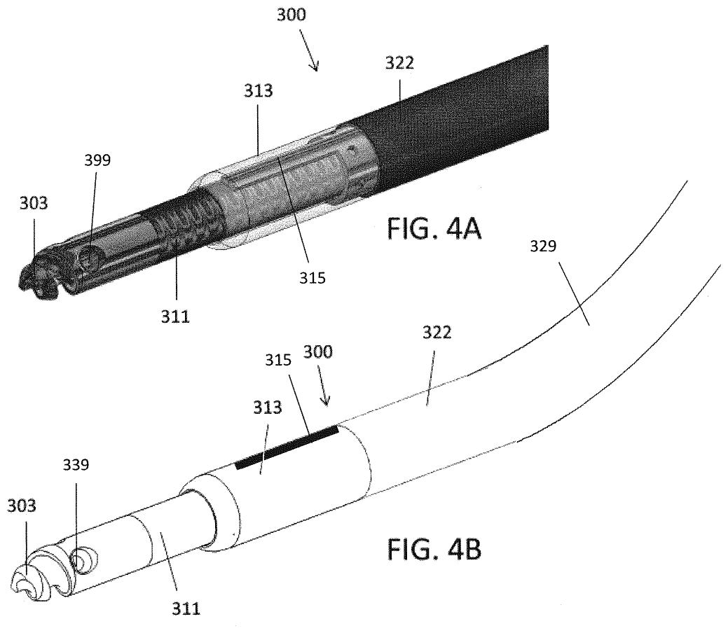

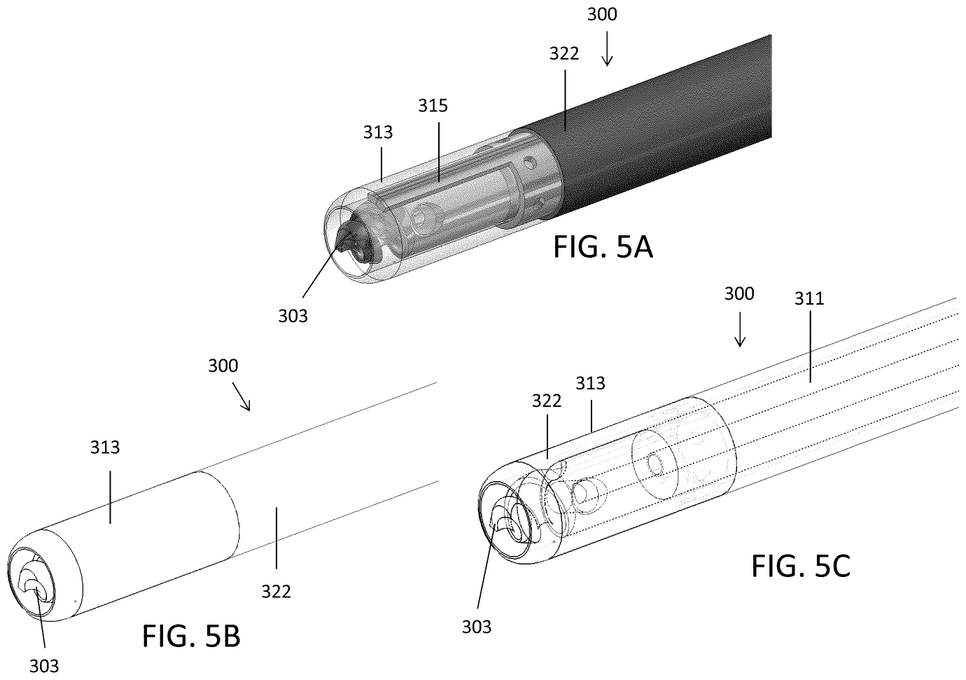

Referring to FIGS. 4A-5C, in another embodiment, an exemplary catheter 300 includes an inner shaft 311, an outer shaft 322, and a distal tip 303 connected to the inner shaft 311. Further, the outer shaft 322 can be separable from the inner shaft 311. For example, the inner shaft 311 can include a luer connector near the proximal end that is attachable and detachable from a luer connector on a proximal end of the outer shaft 322, as described below with respect to handle 900.

In some embodiments, a distal portion 313 of the outer shaft 322 can be clear or transparent, such as made of a clear or transparent plastic, in order to allow imaging therethrough. In some embodiments, the outer shaft 322 can further include a preformed bend 329 therein to help orient or steer the device. A marker 315, such as a metal marker, can extend within the distal portion 313 to indicate the relative orientation of the catheter 300 when in use. For example, as shown in FIG. 4B, the innermost portion of the bend 329 can align with the marker 315.

Further, in some embodiments, the inner shaft 311 can move longitudinally within the hollow outer shaft 322 by sliding a ring on a handle (such as handle 200) connected to the catheter 300 to allow the inner shaft 311 to be exposed (as shown in FIG. 4A-4B) or fully covered (as shown in FIGS. 5A-5C). In use, the inner shaft 311 can thus be extended out of the outer shaft to help drill through the occlusion and pulled in when dissection is not required (or when only blunt dissection is required). In some embodiments, the inner shaft 311 can be configured to be fixed at various points relative to the outer shaft 322 so as to vary the amount of exposed tip 103. Further, the shaft 311 can be fully removed from the outer shaft 322 to allow for placement of a guidewire therethrough.

Further, the device 300 can include an imaging element 399 similar to as described above with respect to device 100. The catheter 300 can be configured to image with the imaging element 399 both when the inner shaft 311 is extended distally out of the outer shaft 322 and when the inner shaft 311 is positioned within the outer shaft 322 (through the transparent distal portion 313).

The device 300 can further or alternatively include any of the features, materials, and/or dimensions described above with respect to device 100.

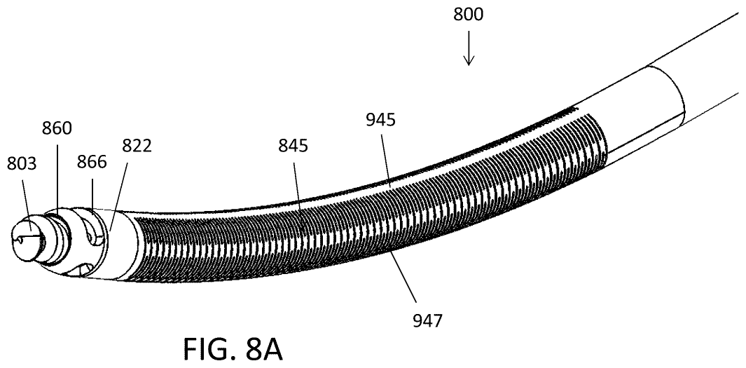

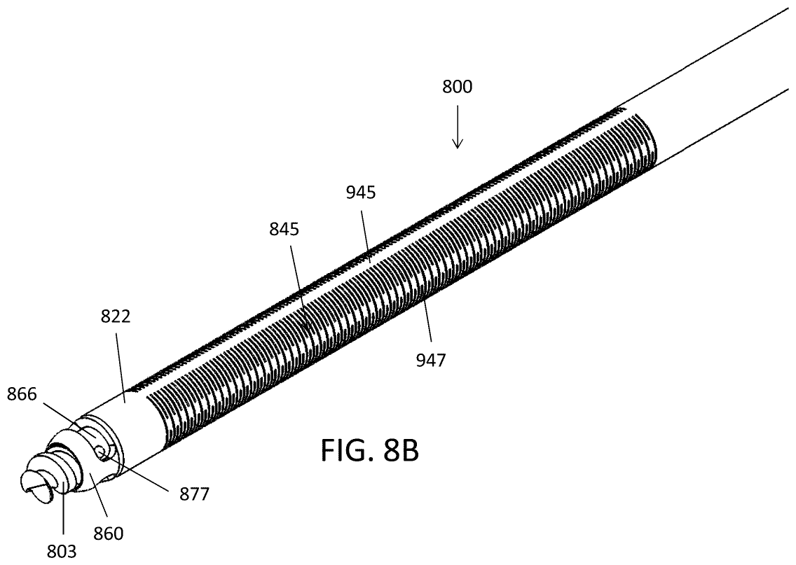

Referring to FIGS. 8A-10, in another embodiment, an exemplary catheter 800 can include both a separable inner shaft 811 and outer shaft 822 and an articulating feature 845 on the distal end of the outer shaft 822.

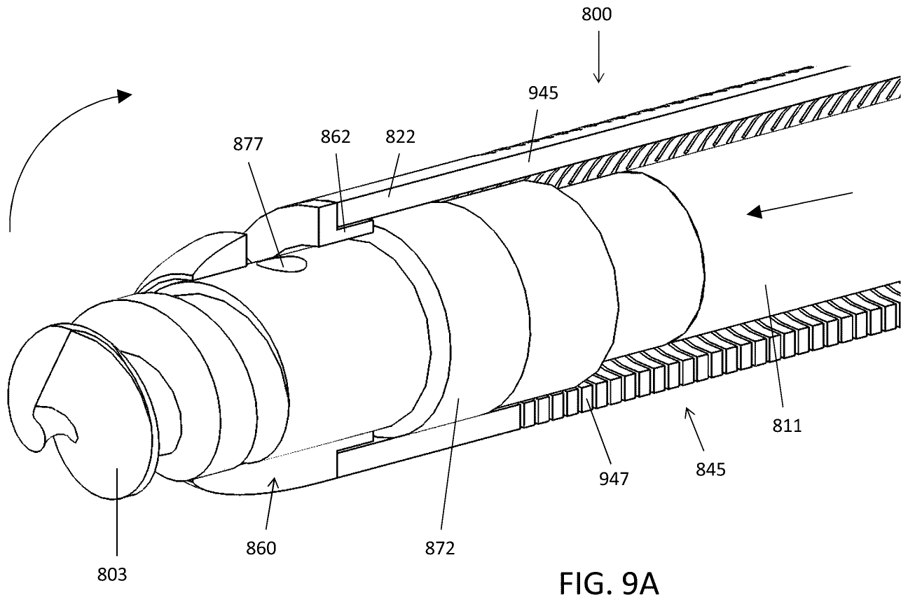

Referring to FIGS. 8A-8B, the articulating feature 845 can include a backbone 945 and a series of circumferential cuts 947. Further, as shown in FIG. 9A, a collar 860 attached to the outer shaft 822 can include an inner ledge 862 configured to extend radially inwards relative to the outer shaft 822. Likewise, the inner shaft 811 can include an annular member 872, such as a plastic bearing, that has a greater diameter than the rest of the inner shaft 811. Thus, when the inner shaft 811 is pushed distally, the annular member 872 of the inner shaft 811 can push against the inner ledge 862 of the collar 860. As a result, the outer shaft 822 can bend at the cuts 947 towards the backbone 945 (as shown by the arrows in FIG. 9A).

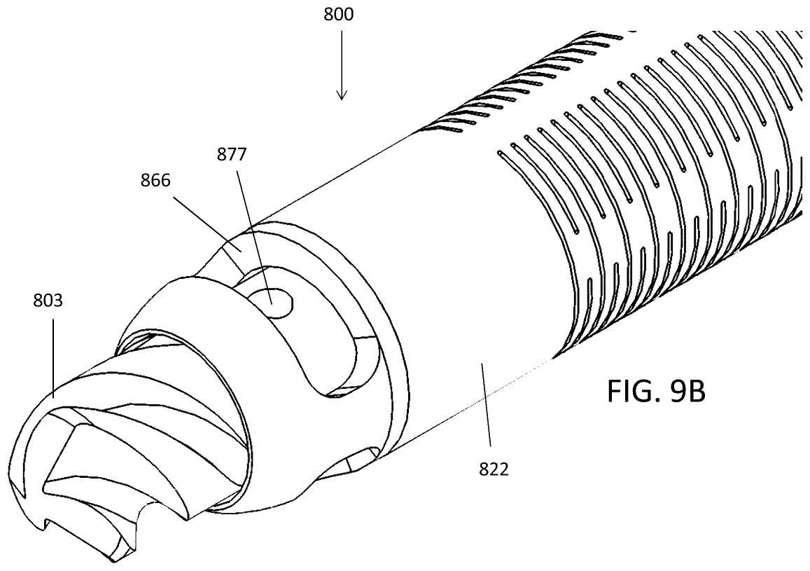

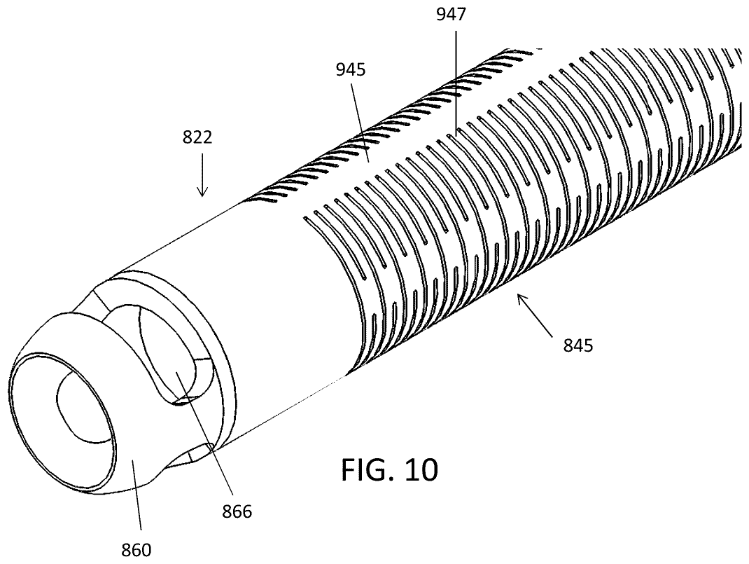

As shown in FIG. 10, the inner shaft 811 can be fully removable from the outer shaft 822 and collar 860 by pulling the inner shaft 811 proximally. By doing so, the outer shaft 822 can be used as a sheath, e.g., for guidewire placement.

Further, the inner shaft 811 can include an imaging element 877 element similar to as described above with respect to devices 100 and 300 that is rotatable with the inner shaft 811. The imaging element 877 can image through imaging windows 866 in the collar 860. Further, the inner ledge 862 can also function to properly align the imaging element 877 with the imaging windows 866 when the inner shaft 811 is within the outer shaft 822.

The inner shaft 811 can include a rotatable distal tip 803 similar to as described above with respect to devices 100 and 300. Likewise, the device 800 can alternatively or additionally include any of the materials and dimensions described above with respect to devices 100 and 300.

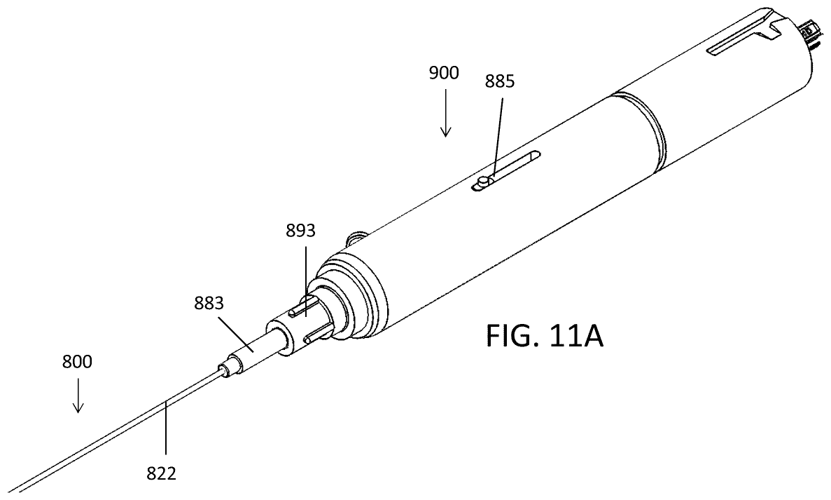

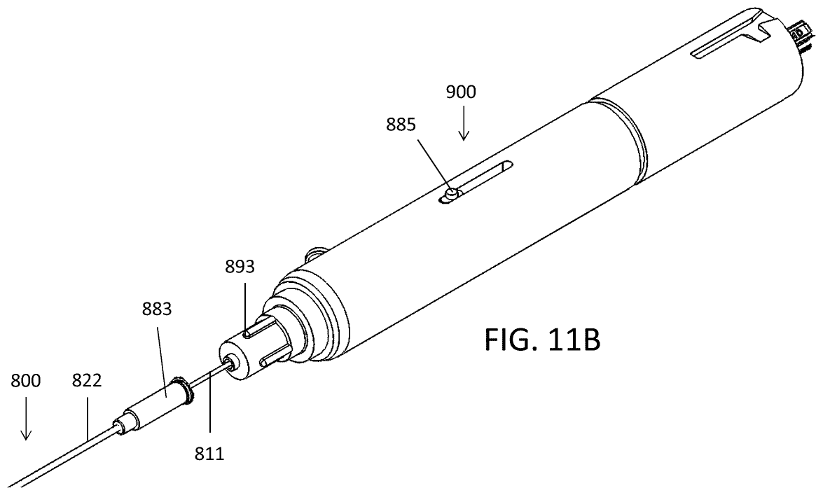

Referring to FIGS. 11A-11B, a handle 900 can be used to operate the device 800. The handle 900 can include a luer lock 883 configured to lock the inner shaft 811 and outer shaft 822 together longitudinally. The luer lock 883 can be configured to provide some relative longitudinal movement between the outer shaft 822 and the inner shaft 811 such that the inner shaft 811 can still move a small distance, such as between about. 125 inches to 0.2 inches, to activate the articulating feature 845. For example, the inner shaft 811 can include an accordion or elastomeric segment to provide the additional relative movement. The actual displacement distance depends on the diameter of the outer shaft of the catheter, the degree of bending that is desired and the elongation/compression of the outer and inner shaft, respectively. The larger the diameter of the outer shaft, the greater the desired degree of bending, and the more compression/elongation of the shafts, the greater the required amount of displacement. Further, the luer lock 883 can be configured to allow the inner shaft 811 to rotate freely within the outer shaft so as to provide rotation of the sharp distal tip 803 connected to the inner shaft 811. The luer lock 883 can be configured such that the outer shaft can be rotated relative to the position of the handle. With the shaft in the articulated position, rotating the outer shaft will direct the catheter around or towards obstacles during use. If the luer lock 883 is disconnected, as shown in FIG. 11B, the inner shaft 811 can be pulled out of the outer shaft 822 by the handle, leaving the outer shaft 822 in place, such as for guidewire placement.

The handle 900 can further include a lever 885 or ring configured to control the axial movement of the inner shaft 811 (and thus the articulation of the device 800). In some embodiments, the lever 885 can include a locking mechanism that allows the device 800 to stay bent at a set angle. The handle 900 can also include a rotation element 893 attached to the outer shaft 822 and configured to rotate the outer shaft 822, such as 360 degrees, to position the bend of the device 800 in the desired orientation.

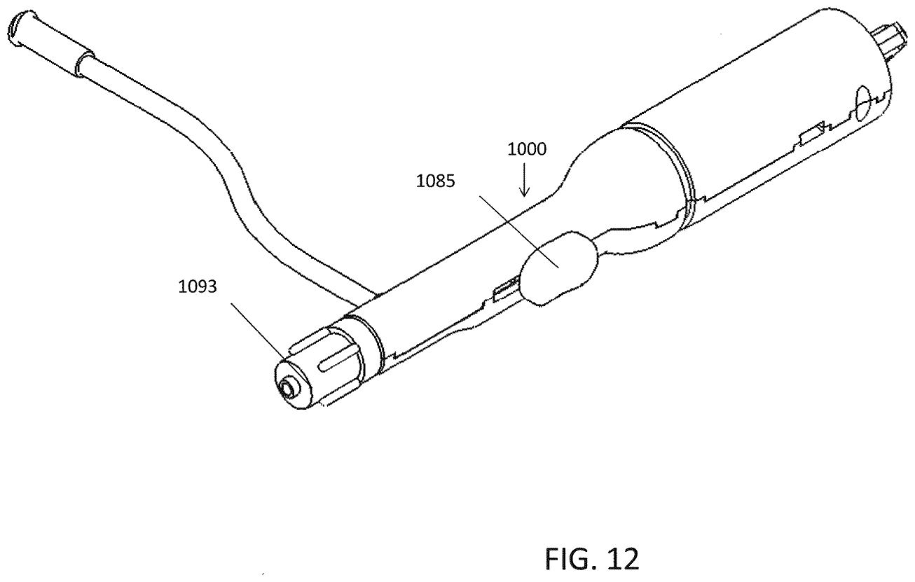

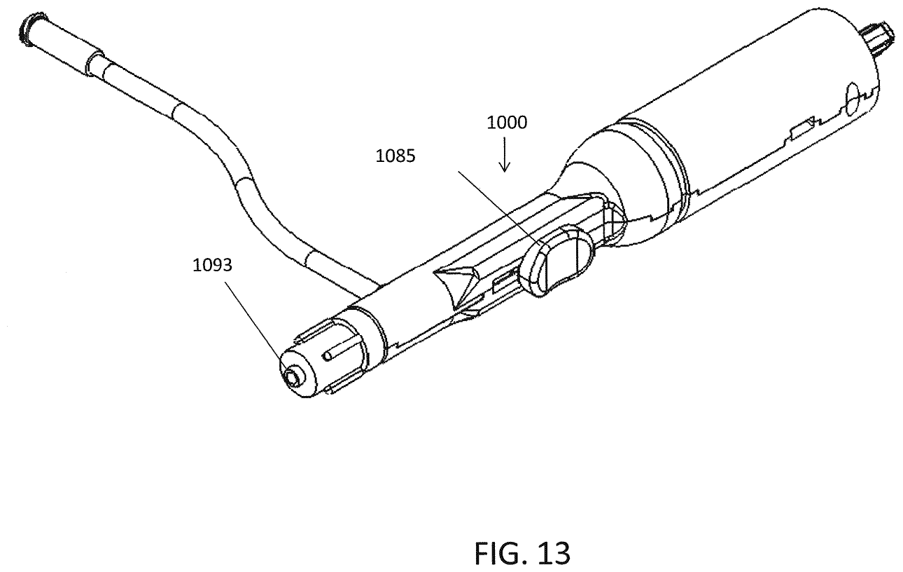

Another exemplary handle 1000 is shown in FIGS. 12-13. The handle 1000 can include many of the features of handle 900. A slide button 1085 can be used to control the axial movement of the inner shaft. The rotation element 1093 can be configured to rotate the outer shaft 822.

Furthermore, in some embodiments, the connection between the outer and inner shafts within the handle can be configured such that the two shaft snap together, axially fixing the proximal ends together, but allowing them to rotate independently. In other embodiments, a third element could be used to key, link, or peg the two shafts together.

Features of the handles 900, 1000, though described for use with catheter 800, can likewise be used with catheters 100, 300.

The distal end of another embodiment of a catheter 1400 is shown in FIGS. 14A-14B. The catheter 1400 is similar to catheters 100, 300, 800 except that it includes a smooth distal tip 103 and a molded distal portion 1410. Thus, the distal tip 103 can have a smooth angled surface 1413 that is non-fluted and comes together in a slightly convex distal point 1415 (i.e., the tip can be frusto-conical). The distal tip 103 of FIGS. 14A, 14B can advantageously provide less aggressive drilling through the occlusion. The distal tip 103 of FIGS. 14A and 14B can be used in place of any of the distal tips described with respect to catheters 100, 300, 800. Likewise, the catheter 1400 can include a molded distal portion 1422. The molded distal portion 1422 can be similar to the distal end of the catheter 300 and can include a bushing 1424, a transparent section 1422, and the scaffolding 1452 of the outer shaft. Further, an imaging fiber 1499 can run down the central axis of the device, as described above with respect to other embodiments. Any of the features of catheter 100, 300, 800 can be used in addition to, or as an alternative to, the features described with respect to catheter 1400. Likewise, the catheter 1400 can be used with a handle having some or all of the features of handles 200, 900, or 1000.

In some embodiments, all or a portion of the outer shaft of the catheters described herein can be clear to allow imaging therethrough. Further, in some embodiments, the catheters described herein can include a balloon to occlude for better imaging. The balloon can be a clear balloon to allow imaging therethrough.

As described above, the catheters 100, 300, 800, 1400 can include an imaging element. The imaging element can include an optical fiber, such as an optical coherence tomography (OCT) imaging fiber. The optical fiber can extend within the driveshaft or inner shaft so as to extend substantially along the central axis of the catheter for the entire length of the fiber. The fiber can be attached at the distal end of the driveshaft or inner shaft and/or the distal tip, but can be otherwise free to float within the driveshaft. The imaging fiber can transfer an OCT signal for imaging of the vessel in which the device is placed. In some embodiments, the imaging fiber can have a polyimide coating therearound within the length of the driveshaft to support and protect the fiber as it spins within the driveshaft. Further, the handles described herein can be configured to accommodate a certain amount of slack in the fiber to facilitate extension and retraction of drive shaft against hollow shaft.

The imaging element can further include a mirror oriented at an angle (such as a 30-60 degree angle, e.g., 45 degrees) with respect to the central axis of the fiber such that light coming out of the fiber will bounce off the mirror and into the adjacent tissue. Glue can be used to hold the distal end of the optical fiber in place. The glue can have a refractive index configured to be appropriately mismatched with the refractive index of the fiber, as described in U.S. patent application Ser. No. 12/790,703, titled “OPTICAL COHERENCE TOMOGRAPHY FOR BIOLOGICAL IMAGING,” filed May 28, 2010, Publication No. US-2010-0305452-A1; and International Patent Application No. PCT/US2013/031951, titled “OPTICAL COHERENCE TOMOGRAPHY WITH GRADED INDEX FIBER FOR BIOLOGICAL IMAGING,” filed Mar. 15, 2013, both of which are incorporated by reference in their entireties. Further, the glue can have a meniscus shape along its outer edge, as described in International Patent Application No. PCT/US2013/031951 titled “OPTICAL COHERENCE TOMOGRAPHY WITH GRADED INDEX FIBER FOR BIOLOGICAL IMAGING,” filed Mar. 15, 2013, incorporated by reference herein. The meniscus shape can advantageously ensure that the light reflected back from the surface of the glue and back into the fiber is significantly less than the light referenced.

The driveshaft or inner shaft, and thus the imaging element or optical fiber, can be configured to rotate continuously at high speeds, such as greater than 500 rpm, greater than 600 rpm, greater than 700 rpm, greater than 800 rpm, greater than 900 rpm, or greater than 1,000 rpm, e.g., between 500-1,000 rpm, in one or both directions to provide OCT imaging around the inner circumference of the vessel. Such high speed rotation in a single direction or in different directions as chosen by the user (as opposed to requiring rotation alternately in both directions to manage the optical fiber) allows for the gathering of image data more quickly, thereby providing more accurate and up-to-date images during use of the device 100. For example, images can be generated at a rate of greater than 6 frames per section (fps), such as greater than or equal to 8 fps or greater than or equal to 10 fps, such as approximately 16.67 fps. In an exemplary embodiment, the rate of Laser sweep, such as approximately 20 KHz, can be configured to keep up with at 16.67 frames per second with about 1200 lines per frame.

Advantageously, because the optical fiber runs through the center of the catheters described herein, the catheters can be small in diameter. For example, the outer diameter of the catheters described herein can be less than 0.10″, such as less than 0.08″, such as less than 0.07″, less than 0.06″, or less than 0.05″. Accordingly, the catheters described herein can advantageously be used in small-diameter peripheral arteries and coronary arteries.

In some embodiments, the catheters described herein can be configured to be attached to a drive system. The drive system can include a rotary optical junction configured to rotate the fiber. Exemplary drive systems that could be used in conjunction with the devices herein are described in U.S. patent application Ser. No. 13/654,357, titled “ATHERECTOMY CATHETERS AND NON-CONTACT ACTUATION MECHANISM FOR CATHETERS,” filed Oct. 17, 2012 and International Patent Application No. PCT/US2013/032089, titled “ATHERECTOMY CATHETER DRIVE ASSEMBLIES,” filed Mar. 15, 2013, each incorporated herein by reference in its entirety.

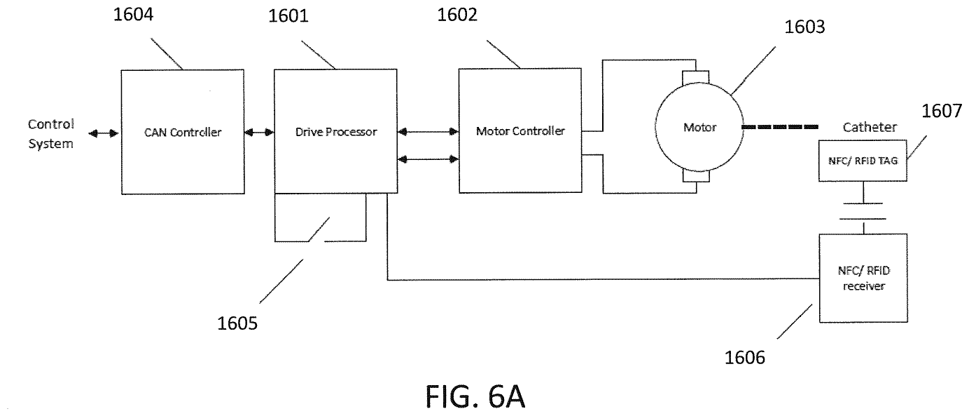

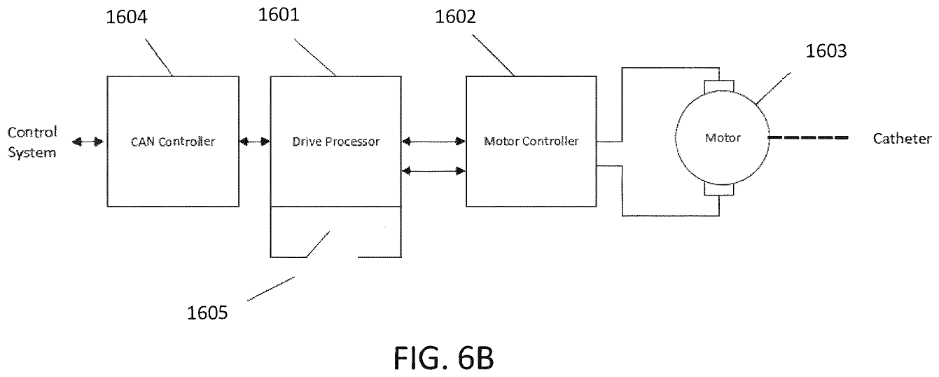

In some embodiments, the drive system can communicate with the control system via a communication bus, which in some embodiments can be a CAN bus 2.0B. This communication can be employed to convey status to the control system or console, such as direction, speed, run status, and other information. It can also be employed to send control information to the drive system, such as run command, speed, direction, and setting of parameters for compensations of mechanical characteristics of the catheters. Referring to FIG. 6A, in one embodiment, a drive processor 1601 is used as the main controlling element for the drive system. The drive processor 1601 controls the motor 1603 through a motor controller 1602, which receives commands and returns status from/to the drive processor 1601. The drive processor 1601 can, in addition to simple speed and direction control, also implement algorithms to optimize catheter performance. The drive processor 1601 communicates with the control system (e.g., the console for the device) via the CAN controller 1604 to send and receive commands and status. In addition, in this embodiment a switch 1605 on the drive processor 1601 housing allows local control of the run state. The switch 1605 can be replaced with alternative hardware inputs, such as buttons, toggles, or knobs.

Further, in some embodiments the drive system can communicate with the catheter via NFC or RFID to obtain information about the catheter. As an example, this information can include catheter type, optimal rotational speed and direction, serial number, amongst many possible parameters. Referring to FIG. 6B, the drive system communicates with the catheter via a NFC/RFID reader 1606 and a NFC/RFID tag 1607 in the catheter to obtain information stored in the tag.

The drive system can be configured to allow the driveshaft and cutter to rotate continuously in the clockwise or the counterclockwise direction depending upon user preference. Therefore, in some embodiments, the drive system can include a user-addressable switch, such as a toggle, to set the desired direction.

Further, in some embodiments, the drive system can include a mechanism to determine the amount of rotation of the driveshaft in the clockwise or counterclockwise directions. Referring to FIGS. 6A and 6B, in one embodiment, for example, the drive system can provide information related to the direction of the motor. Speed and direction can be sensed by the control system (or console) by a data line in the umbilical, which can be a dedicated line or a multiplexed signal. The dedicated line can carry an analog or a digital signal. In one embodiment, a dedicated voltage line carries six discrete velocities (vector speed+direction) that are interpreted by the control system or console in order to infer speed and direction of the catheter.

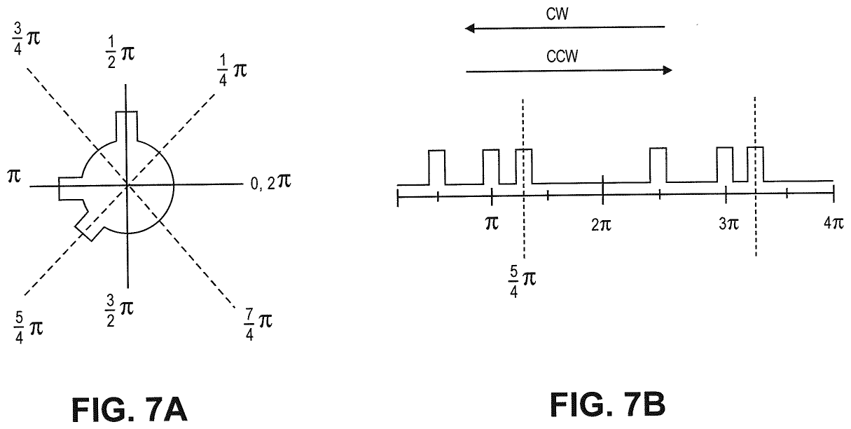

Referring to FIGS. 7A-7B, in on embodiment, a flag in the drive system can include either an asymmetric design or an asymmetric positioning of the flags around the motor (see FIG. 7A). A controller can then sense motor direction by detecting the distinct series of flag spacing and/or width, as shown in FIG. 7B.

Further, in some embodiments, the drive system can be configured to rotate the driveshaft at several discrete rates and/or include a knob to allow for user-chosen continuously variable speeds.

Any of the catheters described herein can be shape-set or include shape-set features to enhance trackability and navigability.

As used herein, an imaging element can include the OCT optical fiber, such as the distal end of the optical fiber, as well as the mirror and adhesive used to hold the mirror and optical fiber in place.

As described above, the catheters described herein can include optical coherence tomography imaging, such as common path OCT. Such OCT systems are described in U.S. patent application Ser. No. 12/829,267, titled “CATHETER-BASED OFF-AXIS OPTICAL COHERENCE TOMOGRAPHY IMAGING SYSTEM,” filed Jul. 1, 2010, Publication No. US-2010-0021926-A1; U.S. patent application Ser. No. 12/790,703, titled “OPTICAL COHERENCE TOMOGRAPHY FOR BIOLOGICAL IMAGING,” filed May 28, 2010, Publication No. US-2010-0305452-A1; and International Patent Application PCT/US2013/031951 titled “OPTICAL COHERENCE TOMOGRAPHY WITH GRADED INDEX FIBER FOR BIOLOGICAL IMAGING,” filed Mar. 15, 2013, all of which are incorporated by reference in their entireties. Alternatively, other types of imaging could be used with the catheters described herein. For example, the devices described herein could be configured to work with infrared spectroscopy or ultrasound.

The catheters 100, 300, 800, 1400 described herein can be used for occlusion-crossing within blood vessels. Advantageously, the devices can advantageously provide increased trackability through bending/steering and high imaging speed during such crossing.

Additional details pertinent to the present invention, including materials and manufacturing techniques, may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are a plurality of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

What is claimed is:

1. An occlusion crossing device comprising:
a rotatable drive shaft having a distal end with a drill tip, wherein the drill tip includes a pointed distal end that allows forward drilling through an occlusion within a blood vessel; and
an outer shaft having a lumen that accommodates the drive shaft, the outer shaft removably coupled to the drive shaft such that drive shaft can be removed from the lumen of the outer shaft when the outer shaft is within the blood vessel.

2. The occlusion crossing device of claim 1, wherein the drill tip includes a plurality of flutes that come together at the pointed distal end of the drill tip.

3. The occlusion crossing device of claim 1, wherein the drive shaft further includes an optical fiber configured to generate images.

4. The occlusion crossing device of claim 3, wherein the optical fiber extends within the drive shaft substantially along a central axis of the drive shaft.

5. The occlusion crossing device of claim 3, wherein a distal tip of the optical fiber is attached to the drill tip.

6. The occlusion crossing device of claim 1, wherein the outer shaft is configured to engage with the drive shaft when a force is applied to the drive shaft in a longitudinal direction to bend the outer shaft.

7. The occlusion crossing device of claim 1, wherein the drive shaft is removably coupled with the outer shaft with a lock that is configured to lock and unlock the drive shaft relative to the outer shaft.

8. The occlusion crossing device of claim 7, wherein the lock is controllable by a proximal handle of the occlusion crossing device.

9. The occlusion crossing device of claim 1, wherein drive shaft includes an annular member that is configured to engage with an inner lip of the outer shaft to bend the outer shaft when the drive shaft is pushed distally.

10. The occlusion crossing device of claim 1, wherein the drive shaft includes an annular member that is configured to engage with an inner lip of the outer shaft to bend the outer shaft when the drive shaft is pulled proximally.

11. The occlusion crossing device of claim 1, wherein the draft shaft is configured to displace longitudinally by a distance of between about 0.125 inches to about 0.2 inches with respect to the outer shaft when a longitudinal force is applied to the drive shaft.

12. A method of crossing an occlusion, the method comprising:
inserting a device into a blood vessel having the occlusion therein, the device having a drive shaft within a lumen of an outer shaft, wherein the drive shaft includes a drill tip with a pointed distal end;
rotating the drive shaft and advancing the device forward such that the pointed distal end of the drill tip drills through the occlusion; and
removing the drive shaft from the outer shaft while the outer shaft is in the blood vessel.

13. The method of claim 12, further comprising inserting a guidewire through the outer shaft after the drive shaft is removed from the outer shaft.

14. The method of claim 12, further comprising collecting images of the blood vessel using an optical fiber attached to the drill tip.

15. The method of claim 14, wherein the images are collected as the optical fiber rotates with the drill tip.

16. The method of claim 12, further comprising bending the outer shaft by applying a force to the drive shaft in a longitudinal direction while the drive shaft is coupled with the outer shaft.

17. The method of claim 16, wherein an inner portion of the outer shaft engages with the drive shaft when the drive shaft is pushed distally.

18. The method of claim 16, wherein an inner portion of the outer shaft engages with the drive shaft when the drive shaft is pulled proximally.

19. The method of claim 12, wherein the draft shaft is displaced longitudinally by a distance of between about 0.125 inches to about 0.2 inches with respect to the outer shaft when a longitudinal force is applied to the drive shaft.

20. The method of claim 12, further comprising unlocking a lock of the device prior to removing the drive shaft from the outer shaft.
